(19) United States
(12) Patent Application Publication
(10) Pub. No.: US 2016/0030472 A1
(43) Pub. Date: Feb. 4, 2016

(54) XENON-BASED ANESTHETIC GAS COMPOSITION USABLE DURING AN ENDARTERECTOMY INVOLVING THE CLAMPING OF THE CAROTID ARTERY

(71) Applicant: Air Liquide Santé (International), Paris (FR)

(72) Inventors: Catherine BILLOET, Suresnes (FR); Yannick LE MANACH, Saint Maur des Fosses (FR)

(21) Appl. No.: 14/883,860

(22) Filed: Oct. 15, 2015

Related U.S. Application Data
(62) Division of application No. 13/820,970, filed on Mar. 5, 2013.

(51) Int. Cl. A61K 33/00; A61K 31/4535; A61K 9/00; A61K 31/454; A61K 31/05; A61K 31/4164; A61K 31/445; A61K 31/4468

(52) U.S. Cl. CPC A61K 33/00; A61K 31/445; A61K 31/4535; A61K 31/4468; A61K 31/454; A61K 31/05; A61K 31/4164; A61K 9/0073

(57) ABSTRACT

A xenon-based anesthetic gas to be used, via inhalation, to maintain or preserve cerebral perfusion during an endarterectomy involving the clamping of the carotid artery in a mammal under general anesthesia. The xenon is preferably used in combination with at least one injectable anesthetic morphine agent such as remifentanil, sulfentanil, fentanyl, and alfentanil. Advantageously, the xenon is mixed with an oxygen-containing gas and administered to the patient after the patient has been anesthetized, put to sleep, and intubated. The use of xenon makes it possible to achieve a reduction in the pressure gradient during the clamping of the internal carotid artery relative to the usual anesthetic agents, and to achieve stable hemodynamics.

### CROSS REFERENCE TO RELATED APPLICATIONS

This application is divisional of U.S. patent application Ser. No. 13/820, 970 filed Mar. 5, 2013, a 371 of International Application PCT/FR2011/052036 filed Sep. 6, 2011 which claims priority to French Application FR 1057587 filed Sep. 22, 2010, the entire contents of which are incorporated herein by reference.

### BACKGROUND

The invention relates to a xenon-gas-based gas medicament which makes it possible, after inhalation, to maintain or preserve, or even improve, cerebral perfusion during an endarterectomy involving the clamping of the carotid artery in a mammal, in particular in a human being, under general anesthesia.

Carotid stenoses or narrowing of the circulatory pathway within the carotid artery develop in hypertensive, diabetic and dyslipidemic patients, and patients who smoke, and frequently accompany coronary artery disease, in particular angina pectoris or a history of infarction, as taught by N. J. Clark et al.; Anesthésie en chirurgie vasculaire [Anesthetia in vascular surgery]; in Miller R D, Eds. Anesthésie [Anesthesia]; Paris; Flammarion, 1996, 1851-1896 or by F Bonnet; Anesthésie en chirurgie vasculaire [Anesthesia in vascular surgery]; in Samii K, Ed. Anesthésie et réanimation chirurgicale [Anesthesia and surgical resuscitation];

Paris; Flammarion 1992: 560-577.

Carotid stenoses thus constitute an obstacle to the vascularization or blood supply irrigating the tissues through the presence of an intra-arterial plaque.

Carotid endarterectomy is the surgical procedure which makes it possible to remove an obstacle in the carotid artery by curettage. It is the technique most commonly used at the current time.

After exposure and clamping of the carotid bifurcation by putting in a clamp, i.e. a type of forceps with long flexible jaws, making it possible to temporarily interrupt the blood circulation, an opening of the artery or arteriotomy is performed on the anterior face of the common carotid artery and extended on the internal carotid artery. The endarterectomy is carried out in a cleavage plane which is located on the external part of the media. The arteriotomy is then closed, either directly or by interposing a prosthetic patch.

A carotid endarterectomy is conventionally performed under general anesthesia. However, such anesthesia poses the double problem of perioperative hemodynamic equilibrium and neurological monitoring during the arterial clamping period, in order to detect ischemia (resulting from an insufficient blood supply) due to the clamping, leading to the production of a cerebral infarction.

Providing efficient cerebral perfusion makes it possible to limit the risk of after-effects in the event of ischemia due to the clamping and to prevent hypoxia and hypercapnia (without extreme hypocapnia) which are responsible for vasodilation and intracranial vascular steal which can cause or accentuate cerebral perfusion deficiencies.

Carotid endarterectomy requires clamping of the internal carotid artery. During the period when the internal carotid artery is clamped, cerebral perfusion is provided by the other vascular axes. Thus, the Willis polygon or cerebral arterial circle is a system of arterial connections located at the base of the brain, allowing a continuous blood supply in order for the brain to function. Indeed, anastomoses between the arteries arriving in the brain make it possible to compensate, within a certain limit, for the insufficiency of an artery.

The Willis polygon is in fact formed, on the one hand, from two internal carotid arteries (right and left), from which are derived the two anterior cerebral arteries (right and left), the latter being joined via the anterior communicating artery (the continuity of the internal carotid arteries forms the middle cerebral arteries or sylvian arteries) and, on the other hand, from a basilar artery, resulting from the fusion of the two vertebral arteries, giving rise to two posterior cerebral arteries (right and left) and also giving rise to two posterior communicating arteries (right and left) which serve to connect the posterior cerebral arteries to the internal carotid arteries.

In order to maintain sufficient cerebral perfusion during the clamping of the internal carotid artery, an increase in systemic systolic arterial pressure (SAP) is usually recommended. To do this, it is common to have recourse to the intravenous injection of vasopressors which narrow the diameter of the arteries, therefore causing an increase in blood pressure.

However, during this surgical procedure, it is observed that the systemic SAP is not equal to the pressure in the Willis polygon, i.e. in the vascular substitute system which enables the brain to receive nutritive blood even if one of the arteries of the neck is damaged or blocked, and a pressure gradient (PG), i.e. a differential between the systemic SAP measured at the level of a radial arterial catheter (wrist artery) contralateral to the surgery and the arterial pressure in the clamped carotid artery, is described. This pressure gradient indicates that the perfusion pressure at the cerebral level is not as good during the anesthesia.

Moreover, the documents Controversies in Carotid Endarterectomy, by R. Samuel, Mar. 27, 2009, Univ. of Kwazulu-Natal, Dept. Anaesthetics; and Carotid Endarterectomy, by S. J. Howell, Brit. J. of Anaesth. (BJA), 99(1); 119-31 (2007), clearly show the controversies that exist in the medical field with regard to procedures of this type and especially the type of anesthesia to be implemented and the anesthetic compounds to be used for this purpose.

Thus, it can be read therein that local anesthetics, compared with general anesthesia, are reputed to less frequently bring about the insertion of shunts to avoid the low-pressure phenomenon linked to clamping, to reduce cardiorespiratory complications and especially to preserve cerebrovascular autoregulation. They supposedly in particular make it possible to be able to increase systemic blood pressure after aortic clamping and to maintain cerebral perfusion in patients. General anesthetics are, moreover, synonymous with high risks of thrombus, of undetected ischemia, etc.

At the current time, the choice of the anesthesia technique, locoregional or general, is not clearly established in the scientific community, each of the methods having its own advantages and drawbacks.

These documents also perform an inventory of the various known anesthesia techniques with volatile agents or injectable agents (TIVA). They conclude that the most promising candidate among the volatile agents for performing an endarterectomy involving the clamping of the carotid artery under general anesthesia is sevoflurane since it makes it possible, at certain concentrations, to increase cerebral blood flow, to maintain static autoregulation and the response of the cerebral circulation to CO₂, and also to provide preconditioning and neuronal protection. Conversely, nitrous oxide is completely inadvisable and xenon is considered to be experimental and liable to confer only neuroprotection.

In any event, these documents report no trial of real use of these various compounds in the context of endarterectomy involving the clamping of the carotid artery, and especially conclude that numerous investigations are still necessary in order to be able to conclude as to the advantage of performing general anesthesia in the context of such a surgical procedure.

On reading these documents, it is easy to understand the dilemma faced by the scientific community with regard to procedures of endarterectomy type involving clamping of the carotid artery.

It therefore follows that the problem which arises is consequently that of being able not only to carry out an effective anesthesia of the patient having to undergo an endarterectomy involving the clamping of the carotid artery under general anesthesia, but also to maintain sufficient peroperative blood perfusion at the cerebral level.

In other words, the problem is that of being able to provide an anesthetic composition comprising an effective, rapidly eliminated anesthetic compound, the use of which is compatible with an endarterectomy involving the clamping of the carotid artery under general anesthesia, i.e. which has little or no hemodynamic effect, in order to reduce the risk of postoperative brain damage caused by a deficiency in peroperative blood perfusion.

### SUMMARY

The solution of the invention is a xenon-based anesthetic gas composition to be used, via inhalation, to maintain or preserve cerebral perfusion during an endarterectomy involving the clamping of the carotid artery in a mammal under general anesthesia.

The solution of the invention is particularly surprising insofar as inhaled xenon makes it possible to maintain/preserve cerebral perfusion during an endarterectomy involving the clamping of the carotid artery in a patient under general anesthesia, although xenon was not known to have this ability in the context of such a procedure. Indeed, as emphasized by the abovementioned documents from the University of Kwazulu-Natal and from the BJA, xenon was reputed to have only a neuroprotective effect, or even to be inadvisable in such a procedure because it was capable of having a negative effect on cerebral perfusion.

The fact of having demonstrated that xenon can be used beneficially as an inhalable volatile anesthetic agent for preserving or maintaining, or even improving, cerebral perfusion during an endarterectomy involving the clamping of the carotid artery under general anesthesia goes against the generally accepted ideas of the scientific community.

The inhaled xenon thus used makes it possible to ensure good hemodynamics and to reduce the risk of postoperative brain damage caused by a deficiency in peroperative blood perfusion.

As appropriate, the anesthetic gas composition of the invention can comprise one of more of the following characteristics:
the xenon in the gas composition is at a concentration of from 50% to 70% by volume;
the xenon is used in combination with at least one injectable anesthetic agent. Preferably, the injectable anesthetic agent is a morphine compound. Advantageously, the morphine compound is chosen from remifentanil, sulfentanil, fentanyl and alfentanil. By way of indication, the dosages for these morphine compounds may be the following: remifentanil (from 0.2 to 0.5 μg/kg/min), sulfentanil (approximately 10 μg bolus), fentanyl (from 0.05 to 1 mg) and alfentanil (from 50 to 100 μg/kg). In fact, the xenon is preferentially always combined with such an injectable anesthetic agent in order to make it possible to combat the pain generated by the surgical procedure in the patient;
the xenon is used as sole hypnotic agent, i.e. the xenon is generally used alone, i.e. without additional hypnotic agent;
alternatively, the xenon is used with an additional hypnotic agent when the patient is “difficult”. In this case, the xenon can be used in combination with a hypnotic anesthetic agent which can be injected intravenously or which can be administered by inhalation. As appropriate, the injectable hypnotic agent is chosen from propofol (dosage of from 1 to 5 mg/kg or target of 1.5 μg/ml) and etomidate (dosage of from 0.15 to 0.4 mg/kg), or the hypnotic agent is inhalable and is chosen from sevoflurane, desflurane and isoflurane (according to their respective minimum alveolar concentrations—MACs);
the mammal is a human being, i.e. a man or a woman, including children, adolescents, or any other group of individuals;
the xenon gas is mixed with an oxygen-containing gas, in particular the xenon is mixed with pure oxygen, an air/O₂ mixture or an N₂/O₂ mixture;
the xenon is administered prior to, simultaneously with and/or after the administration of the injectable anesthetic agent;
the xenon is administered after the administration of the injectable anesthetic agent;
the gas composition contains a volume proportion of xenon of between 50% and 70% by volume, preferably of the order of at least 55% and/or of at most 65% of xenon by volume;
the xenon is mixed with at least 25% by volume of oxygen, preferentially with at least 30% of oxygen;
the administration of xenon gas begins after the patient has been anesthetized by means of the injectable anesthetic agent, preferably when the patient has been put to sleep and intubated;
the administration of xenon gas by inhalation is carried out via an anesthesia ventilator;
the administration of xenon continues throughout the entire duration of the surgery, preferably until the waking and extubation of the patient;
the general anesthesia is induced by the injectable anesthetic agent.

### BRIEF DESCRIPTION OF THE DRAWINGS

FIG. 1 summarizes the results obtained showing that, in the 24 patients included in the working example study, no postoperative neurological event was observed. The average duration of clamping of the internal carotid artery is 22 min±5 min and is not different between the 2 groups (p=0.65).

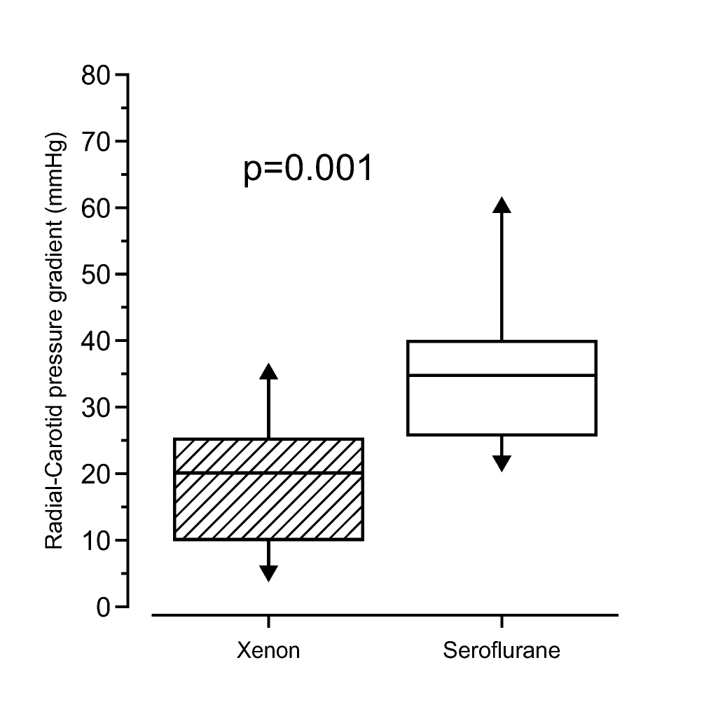

### DESCRIPTION OF PREFERRED EMBODIMENTS

The solution of the invention is therefore based on a use of an anesthetic composition that can be administered to the patient by inhalation, which contains an effective proportion of xenon gas, typically between approximately 50% and 70% by volume, for obtaining and/or for maintaining anesthesia, alone or combined with another anesthetic product. Indeed, xenon inhaled by a patient at a concentration of between 50% and 70% by volume contributes, in combination with one or more morphine products, to maintaining a general anesthesia that makes it possible to perform surgical procedures, in particular an endarterectomy involving the clamping of the carotid artery.

The xenon gas is administered to the patient, once the patient has been put to sleep and intubated, by inhalation via an anesthesia ventilator, for example the Felix Dual™ reference ventilator sold by Air Liquide Medical Systems, in combination with a minimum of 30% by volume of oxygen, throughout the entire duration of the surgery, i.e. until waking and extubation of the patient.

During the maintenance of anesthesia with xenon, it has been possible to note that the hemodynamic parameters are stable, with systemic systolic arterial pressure values that are higher than with the usual inhaled or intravenous (IV) anesthetic agents, which makes it possible, during an endarterectomy involving the clamping of the carotid artery, to ensure good hemodynamics and therefore to reduce the risk of postoperative brain damage caused by a deficiency in peroperative blood perfusion.

Furthermore, the xenon maintains the brain perfusion pressure or prevents a reduction thereof by not causing a major pressure gradient between the systemic circulation and the cerebral circulation.

The xenon gas according to the invention is therefore used to produce an anesthetic gas composition that can be administered by inhalation for maintaining or preserving cerebral perfusion during an endarterectomy involving the clamping of the carotid artery in a mammal under general anesthesia, in particular when this general anesthesia has been induced using a conventional anesthetic agent, in particular an i.v. injectable agent, for example propofol in combination with an i.v.

morphine compound, for example remifentanil.

In other words, the inhalable xenon-based anesthetic gas composition according to the invention can be used in a method for treating a patient who must undergo and endarterectomy involving the clamping of the carotid artery under general anesthesia, comprising the steps of:

a) selecting a patient who must undergo an endarterectomy involving the clamping of the carotid artery under general anesthesia,

b) inducing anesthesia in the patient by means of one (or more) injectable anesthetic agent until general anesthesia of said patient has been achieved and said patient has been put to sleep,

c) intubating the patient,

d) beginning, subsequent to steps b) and c), the administration via inhalation to said patient of a xenon-gas-based gas composition, preferably a gas mixture containing from 50% to 70% by volume of xenon and at least 25% to 30% by volume of oxygen,

e) performing an endarterectomy involving the clamping of the carotid artery in said patient,

f) continuing the administration of xenon during at least step e), preferably until waking and extubation of the patient, so as to maintain or preserve the cerebral perfusion of the patient and to achieve stable hemodynamics.

During steps d) and f), the administration of xenon gas by inhalation is carried out via an anesthesia ventilator, for example the Felix Dual™ ventilator sold by Air Liquide Medical Systems.

The xenon or xenon-based gas mixture constituting the anesthetic gas composition of the invention is preferentially packaged in a gas cylinder under pressure or in liquid form, for example in a cylinder of from one to several liters (water content) and at a pressure of between 2 and 300 bar.

The xenon or xenon-based gas mixture constituting the anesthetic gas composition of the invention can be in “ready-to-use” form, for example premixed with oxygen (30% by volume or more), or else it can be mixed on site at the time of its use, in particular with oxygen and optionally another gas compound, for example nitrogen.

### EXAMPLE

The trials set out hereinafter were carried out in order to demonstrate the positive effects of xenon inhalation in the context of an endarterectomy involving the clamping of the carotid artery under general anesthesia.

The study was carried out in 24 patients scheduled for carotid surgery, comprising men and women.

The choice of treatment of the patients is made randomly (randomization):
either using a routine anesthetic agent, namely sevoflurane: group S of 12 patients,
or by inhalation of xenon at 60%±5% in a mixture with at least 30% of oxygen: group X of 12 patients (% by volume).

Anesthesia was induced identically in the 2 groups using intravenous products, namely propofol (target 1.5 μg/ml) and a morphine compound, remifentanil (target 4 ng/ml), double TCI (Target Controlled Infusion).

The main assessment criterion is the pressure gradient (PG) between the systolic arterial pressure (SAP) measured at the level of a radial arterial catheter contralateral to the surgery and the arterial pressure in the clamped carotid artery. This pressure is obtained by means of a Fogarty catheter, which also enables clamping of the internal carotid artery by inflation of the balloon, connected to a pressure sensor.

The continuous recording and the calculation of the PG are carried out live by a BiopAC system. This method makes it possible to obtain a measurement of the PG throughout the entire duration of the clamping of the internal carotid artery.

The statistical analyses are carried out with R Project for Statistician Computing (www.r-project.org).

The results obtained show that, in the 24 patients included in this study, no postoperative neurological event was observed. The average duration of clamping of the internal carotid artery is 22 min±5 min and is not different between the 2 groups (p=0.65).

The consumption of vasopressor products for maintaining the systemic arterial pressure was significantly higher in the group S compared with the group X (p<0.001).

Furthermore, the average PG of the patients of the group X is significantly lower (p<0.001) than the average PG of the patients of the group S, as can be seen on the appended figure which represents the average radial-carotid pressure gradient (in mmHg) of the patients of the groups S and X.

These results show that the use of xenon for maintaining general anesthesia (GA), during a carotid surgery, thus makes it possible to reduce the pressure gradient (PG) during the clamping of the internal carotid artery compared with the usual anesthesia agents, such as sevoflurane (S) used alone.

The combination of more stable hemodynamics and a reduction in PG are elements in favor of the use of inhaled xenon, in combination with one (or more) injectable anesthetic agent, such as a morphine compound chosen from remifentanil, sulfentanil, fentanyl and alfentanil, for maintaining or improving cerebral perfusion at the time of clamping of the carotid artery during an endarterectomy under general anesthesia.

What is claimed is:

1. A method of maintaining cerebral perfusion during an endarterectomy involving the clamping of the carotid artery in a mammal under general anesthesia comprising the steps of
a) providing an effective amount of a xenon gas composition via inhalation to a mammal under general anesthesia during the endarterectomy involving the clamping of the carotid artery to thereby maintaining cerebral perfusion during the endarterectomy involving the clamping of the carotid artery,
b) providing at least one injectable anesthetic agent to the mammal, and
c) providing at least one hypnotic agent which is injected intravenously or administered by inhalation.

2. The method of claim 1, wherein the injectable anesthetic agent is chosen from remifentanil, sulfentanil, fentanyl and alfentanil.

3. The method of claim 1, wherein the injectable hypnotic agent is chosen from propofol and etomidate.

4. The method of claim 1, wherein the xenon gas composition is administered prior to, simultaneously with and/or after the administration of the anesthetic agent.

5. The method of claim 1, wherein the xenon gas composition is administered prior to, simultaneously with and/or after the administration of the hypnotic agent.

6. The method of claim 1, wherein the xenon gas composition comprises an oxygen-containing gas.

7. The method of claim 1, wherein the xenon gas composition comprises by volume between 50% and 70% of xenon.

8. The method of claim 7, wherein the xenon gas composition comprises by volume between 55% and 65% of xenon.

9. The method of claim 1, wherein the xenon gas composition comprises by volume at least 25% of oxygen.

10. The method of claim 9, wherein the xenon gas composition comprises by volume at least 30% of oxygen.

11. The method of claim 1, wherein the mammal is a human being.

12. The method of claim 1, wherein the injectable anesthetic agent upon injection results in the general anesthesia.

13. The method of claim 1, wherein the xenon gas composition comprising the step of delivering the xenon gas composition for inhalation to the patient from an anesthesia ventilator.

14. The method of claim 12, wherein the step of providing an effective amount of a xenon gas composition via inhalation begins after the mammal has been anesthetized by the injectable anesthetic agent.

15. The method of claim 1, wherein the step of providing an effective amount of a xenon gas composition via inhalation is continued throughout the entire duration of the endarterectomy involving the clamping of the carotid artery.
